(19) United States
(12) Patent Application Publication
(10) Pub. No.: US 2010/0256765 A1
(43) Pub. Date: Oct. 7, 2010

(54) Spinal Implants and Deployment Instruments For Covering Traumatized Spinal Disc Areas

(76) Inventors: Michael S. Butler, St. Charles, IL (US); Thomas J. Wegrzyn, III, Cambridge, MA (US)

Correspondence Address: Bruce J. Bowman;Bowman & Associates, Suite 106, 1016 3rd Ave. SW, Carmel, IN, 46032, US

(21) Appl. No.: 12/752,287

(22) Filed: Apr. 1, 2010

Related U.S. Application Data
(60) Provisional application No. 61/165,711, filed on Apr. 1, 2009.

(51) Int. Cl. A61F 2/44; A61B 17/00

(52) U.S. Cl. 623/17.16; 606/151

(57) ABSTRACT

A spinal implant and method of implant use are provided for application about a spinal implant area. The spinal implant is formed of a resilient and/or elastic covering, and first and second bone fasteners for holding the covering in place. An all-in-one deployment instrument is also provided that delivers and installs the spinal implant to the spinal implant site. The spinal implant provides a covering for the spinal implant site such as a spinal disc fissure, about a portion of a spinal disc after a full or partial discectomy or other procedure, and/or over any spinal disc area. The covering is disposed between and held by the first and second bone fasteners. An application instrument for introducing and installing the present spinal implant is also provided. The instrument introduces then applies the covering at the spinal implant site and drives the vertebral body staples into the vertebral body to secure the covering to the spinal implant site. The covering may include a spinal medicament and/or provide a spinal medicament delivery system.

### RELATED APPLICATIONS

This patent application claims the benefit of and/or priority to U.S. Provisional Patent Application Ser. No. 61/165,711 filed Apr. 1, 2009, entitled “Spinal Implant For Covering A Spinal Disc Fissure And Deployment Apparatus Therefor” the entire contents of which is specifically incorporated herein by this reference.

### BACKGROUND OF THE INVENTION

1. Field of the Invention

The present invention relates to materials, devices and/or implants regarding the spine and/or for use in spinal surgery, and particularly, but not necessarily, pertaining to implants regarding a spinal disc.

2. Background Information

There are many instances due to injury/damage, wear and tear, disease and/or the like that cause the hard outer layer (i.e. anulus fibrosis) of a disc of the spine to tear or crack. When this happens, the gelatinous center (i.e. nucleus pulposus) of the disc can be forced out through the tears, cracks or fissures in the anulus fibrosis. This causes the disc to bulge, break open (rupture), or break into pieces. This is generally known as a herniated disc. Additional terms such as ruptured disc, torn disc (or disc tear), slipped disc, collapsed disc, disc protrusion, or disc disease may also be used. A herniated disc may occur in any part of the spine such as the neck (cervical), the upper back (thoracic) and, the lower back (lumbar).

When a herniated disc bulges out from between the vertebrae, the spinal nerves and spinal cord can become pinched. While there is normally a small amount of space around the spinal cord and spinal nerves, if enough of the herniated disc is pushed out of place (i.e. protruding from the disc), the spinal cord and spinal nerve structures may be compressed. As such, a herniated disc may cause backache, pain, numbness, and weakness in the area of the body where the nerve travels, typically in the buttock and down the leg (known as sciatica).

Treatment for a herniated disc may include surgery. Spinal surgery may be performed to fix or repair the herniated disc or to remove it. Such surgery is generally known as a discectomy. When aiming to fix or repair a herniated disc, a discectomy results in the removal of tissue that was protruding from fissures in the anulus pulposa of the herniated disc. Thereafter, it is necessary to “bandage” or provide support to the operated disc area (i.e. fissures) in order to promote healing and/or prevent further nucleus pulposus leakage or rupture (herniation).

Given the above, it would be desirable to have a manner of covering a fissure of a spinal disc having undergone a discectomy.

Given the above, it would be further desirable to have a manner of providing support to a fissure area of a spinal disc having undergone a discectomy.

Given the above, it would be even further desirable to have a manner of aiding in the prevention of further nucleus pulposus herniation in a post-discectomy spinal disc.

### SUMMARY OF THE INVENTION

A spinal implant and method of implant use are provided for application around (e.g. covering) a spinal disc and/or spinal disc area. The spinal implant is formed of a resilient and/or elastic covering formed as a mesh, netting, fabric or the like and first and second bone fasteners for holding the covering in place. A deployment instrument is also provided that can secure the spinal implant to an implant site.

The spinal implant provides a covering for an implant site such as a spinal disc fissure, about a portion of a spinal disc after a full or partial discectomy or other procedure, and/or over any spinal disc area. The spinal implant includes first and second fasteners and a resilient or elastic covering disposed between and held by the first and second fasteners. In one form, the first and second fasteners are vertebral body staples. An applicator or deployment instrument for the spinal implant is also provided that applies the covering at the spinal implant site and drives the vertebral body staples into the vertebral body to secure the covering to the spinal implant site.

In one form, the vertebral body staples are vertebral bone staples which are configured for reception and retention in a vertebra and/or endplate thereof (collectively, vertebral body). The spinal implant may be attached by one or more staples to a vertebra or one or more adjacent vertebrae. The deployment instrument is fashioned to install the spinal implant. Particularly, the deployment instrument allows 1) the placement of the implant covering over the intended implant covering area, and 2) the securing of the vertebral body staples to the vertebral body and relative to the implant covering in order to retain the implant covering relative to the intended implant covering area.

A vertebral body staple is formed as a generally U-shaped member defined by two legs joined via a crossbar. The legs are configured to be anchored into the vertebral body while the crossbar is configured to retain the implant covering. Length of the crossbar defines width of the staple (i.e. the distance between the two legs). The present vertebral body staples may be made in narrow through wide widths. Length of the legs determines vertebral body penetration depth.

Each leg of a staple has an end that defines a configured tip. The configured tip is formed to pierce and penetrate the vertebral body for anchoring the leg into the vertebral body. In one form, the configured tip is shaped as a conical point, while in another form the configured tip is shaped as an angled chisel. Other tip variations and/or shapes are contemplated and may be used.

Additionally, one or both legs may include configurations formed to aid in anchoring the legs, and thus the staple, to the vertebral body. The leg configurations are preferably, but not necessarily, formed integral with the leg/staple. In one form, the leg configurations may be formed on the inside area of a leg such as by one or more teeth, serrations, juts, ledges, cutouts or the like. In another form, the leg configurations may be formed about the area of the leg such as by conical sections, tapered annular ledges, juts, serrations cutouts or the like. Other configurations and/or shapes are contemplated and may be used. Given the above, it can be appreciated that the present vertebral body staple may thus be formed in various dimensions for various applications.

The resilient and/or elastic implant covering may be a mesh, netting, fabric, man-made or natural material that may or may not be woven such as is bio-compatibly suitable for the present application. In one form, the covering is made from polyester such as polyethylene terephthalate (PET). In addition to PET, the covering may be formed of another biocompatible material, including, but not limited to, polypropylene or polytetrafluoroethylene. All coverings provide resiliency, flexibility, elasticity and/or fluid porosity at the surgical site.

The implant covering may be secured to the first and second fasteners prior to implanting such as by adhesive, stitching, mechanical, or other means. The implant covering may be held in place by the staples.

The implant covering may also be used along with a medicament delivery system while in situ, whereby the covering is either impregnated with a medicament or medicaments, or is mechanically utilized to release medicament(s) from the mesh, or as a primary delivery vehicle for the medicament. Such medicaments, therapeutic agents, include but are not limited to, one or more of the following: culture media, growth factors, differentiation factors, morphogenic proteins, hydrogels, polymers, antibiotics, anti-inflammatory medications, immunosuppressive medications, therapeutically enhanced cells, genetic agents, stem cells, resorbable culture medium, tissue growth or differentiation factors (recombinant generated morphogenetic proteins, PDGF, TGF-.beta., EGF/TGF-.alpha., IGF-I, .beta.FGF), hydrogels, resorbable or nonresorbable synthetic or natural polymers (collagen, fibrin, polyglycolic acid, polylactic acid, polytetrafluoroethylene, etc.). The implant covering may also or additionally be formed of a bioresorbable material in addition to providing medicament(s) delivery and/or any other uses.

### BRIEF DESCRIPTION OF THE DRAWINGS

The above mentioned and other features and objects of this invention, and the manner of attaining them, will become more apparent and the inventions will be better understood by reference to the following description of embodiments of the invention taken in conjunction with the accompanying drawings, wherein:

FIG. 1 is a perspective view of an exemplary embodiment of a spinal disc fissure covering implant or device fashioned in accordance with the present principles;

FIG. 2 is a perspective view of another exemplary embodiment of a spinal disc fissure covering implant fashioned in accordance with the present principles;

FIG. 3 is a perspective view of an exemplary embodiment of an apparatus for deploying, implanting and/or installing a spinal disc fissure covering implant as provided herein;

FIG. 4 is an enlarged perspective view of the tips or ends of the deployment apparatus of FIG. 3;

FIG. 5 is a perspective view of a portion of a human spine showing two adjacent vertebrae with another exemplary embodiment of a spinal disc fissure covering implant fashioned in accordance with the present principles;

FIG. 6 is a perspective view of another exemplary embodiment of an apparatus for deploying, implanting and/or installing a spinal disc fissure covering implant as provided herein;

FIG. 7 is another perspective view of the apparatus of the deployment apparatus of FIG. 6;

FIG. 8 is an enlarged perspective view of the tip or end of the deployment apparatus of FIG. 6;

FIG. 9 is an enlarged perspective view of another exemplary embodiment of a fastening device for the spinal disc fissure covering implants of the present invention;

FIG. 10 is an enlarged perspective view of a further exemplary embodiment of a fastening device for the spinal disc fissure covering implants of the present invention;

FIG. 11 is an enlarged perspective view of a yet further exemplary embodiment of a fastening device for the spinal disc fissure covering implants of the present invention;

FIG. 12 is a perspective view of another exemplary embodiment of an apparatus for deploying, implanting and/or installing a spinal disc fissure covering implant as provided herein, the deployment instrument having a spinal disc fissure covering implant thereon ready for installation;

FIG. 13 is an enlarged perspective view of the tip or end of the deployment instrument of FIG. 12 shown without a spinal disc fissure covering implant thereon;

FIG. 14 is an enlarged perspective view of the tip of the deployment instrument of FIG. 12 showing the mesh portion of a spinal disc fissure covering implant thereon ready for installation; and

FIG. 15 is the enlarged perspective view of FIG. 14 showing a fastener of the spinal disc fissure covering implant protruding from the mesh as part of the installation of the spinal disc fissure covering implant installation.

Like reference numerals indicate the same or similar parts throughout the several figures.

A description of the features, functions and/or configuration of the present invention depicted in the various figures will now be presented. It should be appreciated that not all of the features of the spine plates of the figures are necessarily described. Some of these non discussed features as well as discussed features are inherent from the figures. Other non discussed features may be inherent in component geometry and/or configuration. Moreover, the drawings are not necessarily to scale and certain features may be exaggerated in order to better illustrate and explain the present invention.

### DETAILED DESCRIPTION OF THE INVENTION

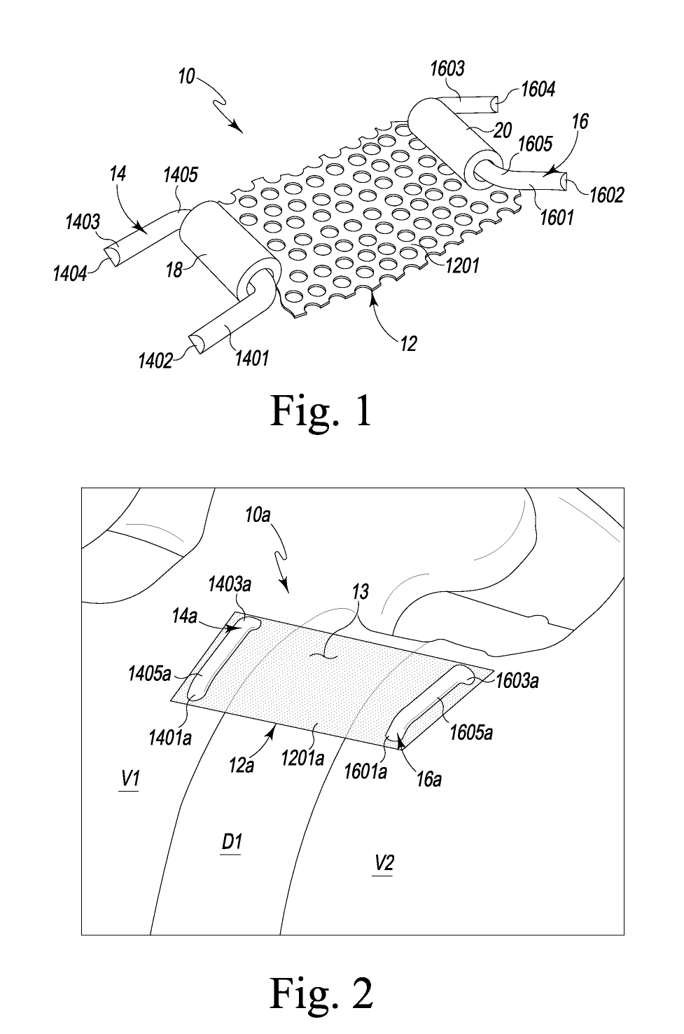

Referring to FIG. 1 there is shown an exemplary embodiment of a spinal implant or device 10 fashioned in accordance with the present principles for the covering of or placement over an intended spinal implant site or area of a spine, such as a fissure of a spinal disc or an area having undergone a spinal surgery. As an example, the spinal implant 10 may be used on a spinal disc having a fissure that has not undergone a discectomy or on a spinal disc having a fissure that has undergone a discectomy. No matter for what purpose, the present spinal implant 10 consists of a covering, layer, overlay or the like defined by a mesh, net, web, lattice, netting, fabric or the like 12 (collectively, “covering”) suspended, held, retained and/or restrained between a first fastener 14 and a second fastener 16. The first and second fasteners 14, 16 are preferably, but not necessarily, fashioned as bone staples such as shown in FIG. 1, but may take other forms as appropriate. It should be appreciated that the bone staples 14, 16 may be identical as shown in FIGS. 1 and 2, but may also be different depending on the application.

The bone staples 14, 16 are fashioned for reception in one or more vertebrae. Particularly, each bone staple 14, 16 is fabricated to be attached, anchored, affixed or fastened to one or more vertebral bodies/endplates (vertebrae) as appropriate. With respect to a fissure, particularly on the superior and inferior ends of the fissure. The bone staples 14, 16 are made from a biocompatible material such as an implantable grade titanium alloy (e.g. Ti 6Al-4V ELI) and are formed as generally U-shaped members.

Bone staple 14 is defined by a crossbar 1405 with a first leg 1401 and a second leg 1403, the first and second legs 1401, 1403 being generally transverse to the crossbar 1405. The first and second legs 1401, 1403 and the crossbar 1405 are formed having a generally annular cross section.

The first end 1402 of the first leg 1401 has an angled tip defining a chisel tip. The point of the chisel tip 1402 is angled so as to be a lateral point or edge. Likewise, the second end 1404 of the second leg 1403 has an angled tip defining a chisel tip. Again, the point of the chisel tip 1404 is angled so as to be a lateral point or edge. Other configurations are contemplated and capable of use.

Bone staple 16 is defined by a crossbar 1605 with a first leg 1601 and a second leg 1603, the first and second legs 1601, 1603 being generally transverse to the crossbar 1605. The first and second legs 1601, 1603 and the crossbar 1605 are formed having a generally annular cross section.

The first end 1602 of the first leg 1601 has an angled tip defining a chisel tip. The point of the chisel tip 1602 is angled so as to be a lateral point or edge. Likewise, the second end 1604 of the second leg 1603 has an angled tip defining a chisel tip. Again, the point of the chisel tip 1604 is angled so as to be a lateral point or edge. Other configurations are contemplated and capable of use.

The covering 12 is made from a body 1201 of a biocompatible, generally resilient and/or elastic material such as a polyester and particularly, but not necessarily, polyethylene terephthalate (PET). The covering 12 is also preferably, but not necessarily, woven. Other biocompatible materials, both natural and man-made and/or covering configurations may be used. The covering may be a rigid or mesh type polyglycolic or polylactic acid that provides a shell covering that is resorbable by the body over time.

In the embodiment shown in FIG. 1, the spinal implant 10 is formed as a pre-assembled implant. Particularly, the covering 12 is pre-attached to each bone staple 14, 16. One end of the covering 12 is retained or held onto the bone staple 14 by a clip or similar device 18 that extends about the crossbar 1405 of the bone staple 14, while the other end of the covering 12 is retained or held onto the bone staple 16 by a clip or similar device 20 that extends about the crossbar 1605 of the bone staple 16. The clips 18, 20 fix the covering relative to the bone staples 14, 16. In use, the covering 12 is stretched taught over an intended spinal disc area (implant area such as a spinal disc fissure) and held in place by the bone staples 14, 16. In this manner, the covering 12 is held taught by and between the bone staples 14, 16 and provides a cover, covering, layer or overlay over the implant area. It also provides support to the annulus and preventing the nucleus pulposus from protruding from the spinal disc onto a nerve structure when so implanted.

In FIG. 2 there is depicted a version of the spinal implant 10 of FIG. 1 (labeled FIG. 10a in FIG. 2) and which is shown implanted or deployed relative to a spinal disc D1 that is disposed between adjacent vertebrae V1 and V2 of a spine and, particularly, relative to a fissure or crack 13 (representing one or more fissures or cracks) in the disc D1. The disc D1 may or may not have undergone a discectomy or other spinal disc procedure. The spinal implant 10a consists of first and second bone staples 14a, 16a and a covering 12a. The covering 12a is like covering 12 described above but is shown disposed over or covers the fissure 13 in the disc D1. The first and second bone staples 14a, 16a are like bone staples 14, 16 described above, but do not include a clip or other mesh fastening device. Rather, the body 1201a is captured beneath or under the respective crossbars 1405a, 1605a of bone staples 14a, 16a, while leg pairs 1401a, 1403a and 1601a, 1603a of bone staples 14a and 16a, respectively, are anchored, implanted, affixed, installed, attached or otherwise connected to the vertebral bodies. In this manner, the covering 12a is held, retained or captured taught over the fissure 13 and between the staples 14a, 16a.

It should be appreciated that other manners or methods of holding or retaining the covering relative to a staple may be used. For instance, the ends of the covering may be formed into each one of the staples. The covering may alternately be adhered to each staple, or threaded, stitched or similarly fastened to the staples. Other manners of holding the covering relative to the staples, as well as other types of clips for holding the covering onto the staples are envisioned.

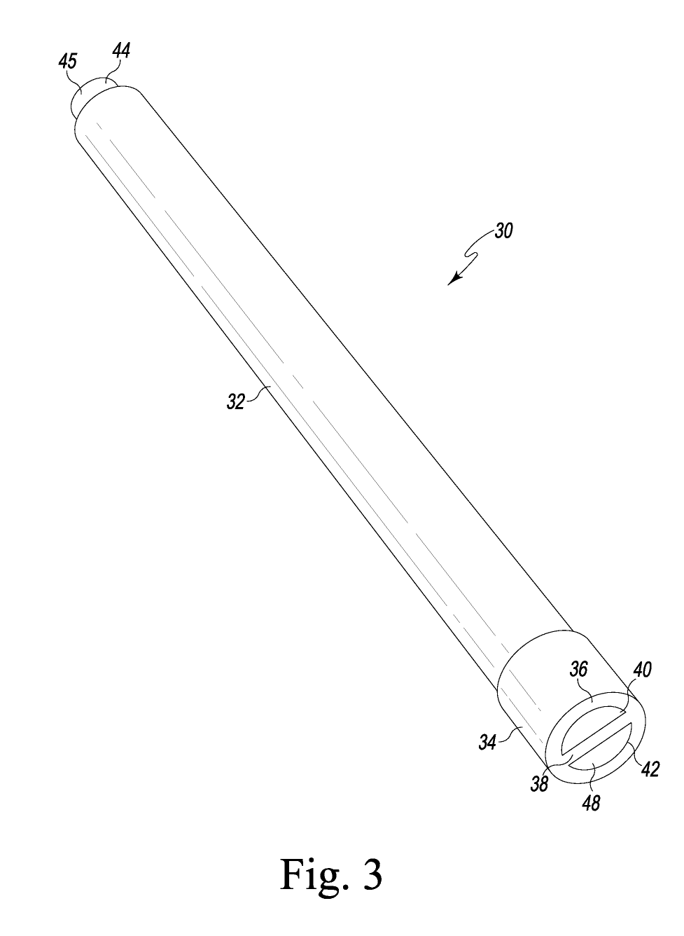

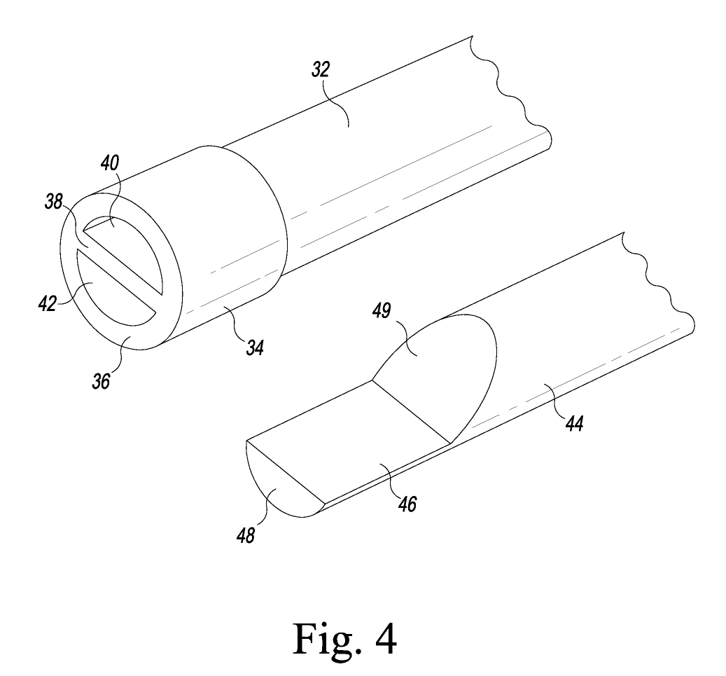

Referring now to FIGS. 3 and 4, there is depicted an exemplary embodiment of an instrument, device or apparatus, generally designated 30, for deploying, placing, installing and/or implanting (collectively, “deployment instrument 30”) the spinal implants of the present invention. The deployment instrument 30 is configured, adapted and/or operable to place, install or implant the bone staples 14/14a, 16/16a or other bone fasteners of the spinal implants 10/10a or similar spinal implant fashioned in accordance with the present principles, into a vertebrae (e.g. V1 and V2) or into an endplate of a vertebrae. The deployment instrument 30 is formed of a suitable material such as a metal and, particularly but not necessarily, a titanium based metal.

The deployment instrument 30 is defined by a handle 32 and plunger or driver 44. The handle 32 carries the staples for implanting while the plunger 44, aided by an externally applied mallet or the like, deploys the staples (i.e. implants them into the vertebrae/vertebrae endplate). The handle 32 is defined by a hollow tube that is open at one end to receive the plunger 44, and which has an end structure 34 on the other end thereof. The end structure 34 includes a first opening 40 and a second opening 42 separated by a middle structure 38. The first and second openings 40, 42 are each configured to receive and hold a staple therein (with the legs of a staple extending axially out of corners of the particular opening) and to allow the staple to be driven out of the opening by the plunger 44. The two openings 40, 42 allow for two staples to be held and implanted. The end structure 34 may have more or less openings, the number of which corresponds to the number of staples (or other fasteners) that may be implanted by the deployment instrument 30.

The plunger 44 is defined by a tubular (preferably, but not necessarily solid) rod that is sized to be received in the hollow handle 32. The plunger 33 is rotatable and axially movable in and relative to the handle 32. The end 45 of the plunger 44 is configured to allow striking thereof for driving a staple held by the end structure 34 into the vertebra. Striking of the end 45 of the plunger 44 while holding the handle 32, axially moves the plunger 44 relative to the handle 32 to impact against a staple for driving the staple into the vertebra. As best seen in FIG. 4, the other end of the plunger 44 has a flat 46 defined adjacent a taper 49 of the plunger 44 and defining a driving end 48. The driving end 48 is configured in similar manner to an opening (40, 42) in order to fit therein and extend therethrough for contacting (impacting) and driving a staple from the opening into a vertebra. Rotation of the plunger 44 rotates the flat 46 and thus the driving end 48 into alignment with an opening 40, 42. The driving end 48 can be seen in FIG. 3 within the opening 42. It can be appreciated that the staple may or may not have a covering associated therewith.

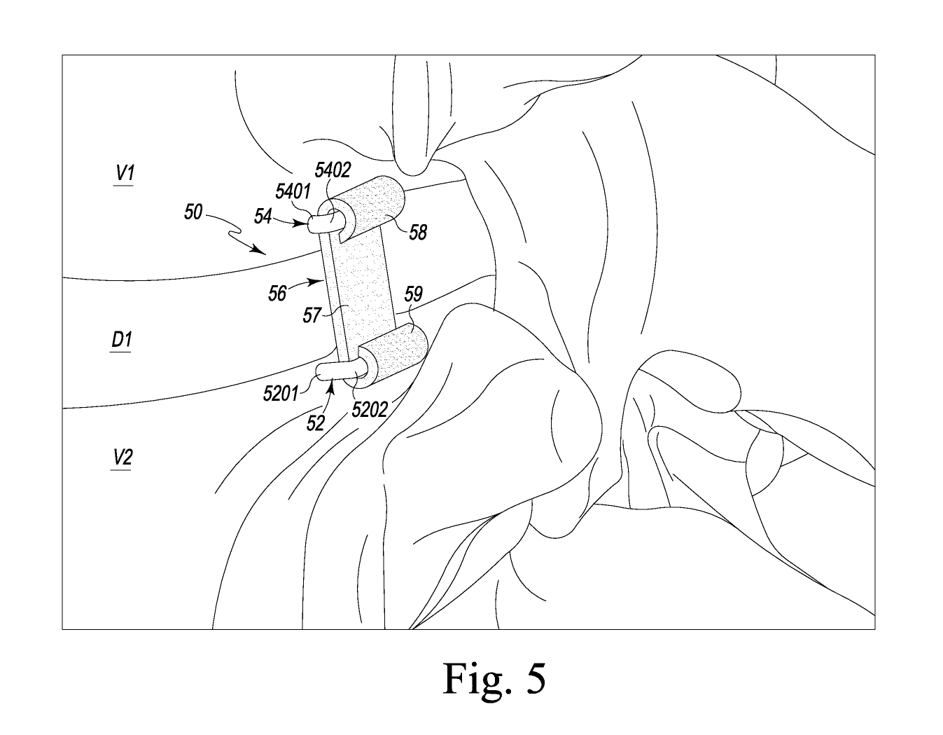

Referring now to FIG. 5, there is depicted another exemplary embodiment of a spinal implant, generally designated 50, fashioned in accordance with the present principles and shown deployed relative to the spinal disc D1. The spinal implant 50 is defined by a first fastener 52, a second fastener 54, and a cover/covering 56. The first fastener 52 is configured as a bone staple made from a suitable biocompatible material, such as titanium, and includes a first leg 5201, a second leg (not seen), and a connecting crossbar 5202. The second fastener 54 is likewise configured as a bone staple made from a suitable biocompatible material, such as titanium, and includes a first leg 5401, a second leg (not seen), and a connecting crossbar 5402. Other biocompatible materials and fastener configurations may be used and are envisioned. The first fastener 52 is shown implanted in the lower or inferior vertebra or endplate thereof V1, while the second fastener 54 is shown implanted in the upper or superior vertebrae or endplate thereof V2. The covering 56 is retained or held by and between the first and second fasteners 52, 54.

The covering 56 is preferably made from a biocompatible, generally elastic material such as a polyester (e.g. PET). Rather than being a mesh as described above, the covering 56 may be sponge-like, gel-like or cellular in form. The covering 56 could be rigid in form such as a plate or the like. The mesh may be a rigid or mesh type polyglycolic or polylactic acid that provides a shell covering that is resorbable by the body over time. However, general resiliency and/or elasticity of the material should allow for the covering 56 to be retained or stretched taught over a fissure in the spinal disc D1 (or other spinal implant site or area). The covering 56 defines a contact or intermediate portion 57 that overlays or covers a fissure (not seen) in the spinal disc D1 or other spinal implant site or area. The covering 56 also defines a first (inferior) end 59 and a second (superior) end 58 of the intermediate portion 57. The first (inferior) end 59 of the intermediate portion 57 extends under and over (about) the crossbar 5202 of the inferior (first) fastener 52, while the second (superior) end 58 of the intermediate portion 57 extends under and over (about) the crossbar 5402 of the superior (second) fastener 54. The covering 56 may be inserted with a posterior approach through a hemilaminectomy.

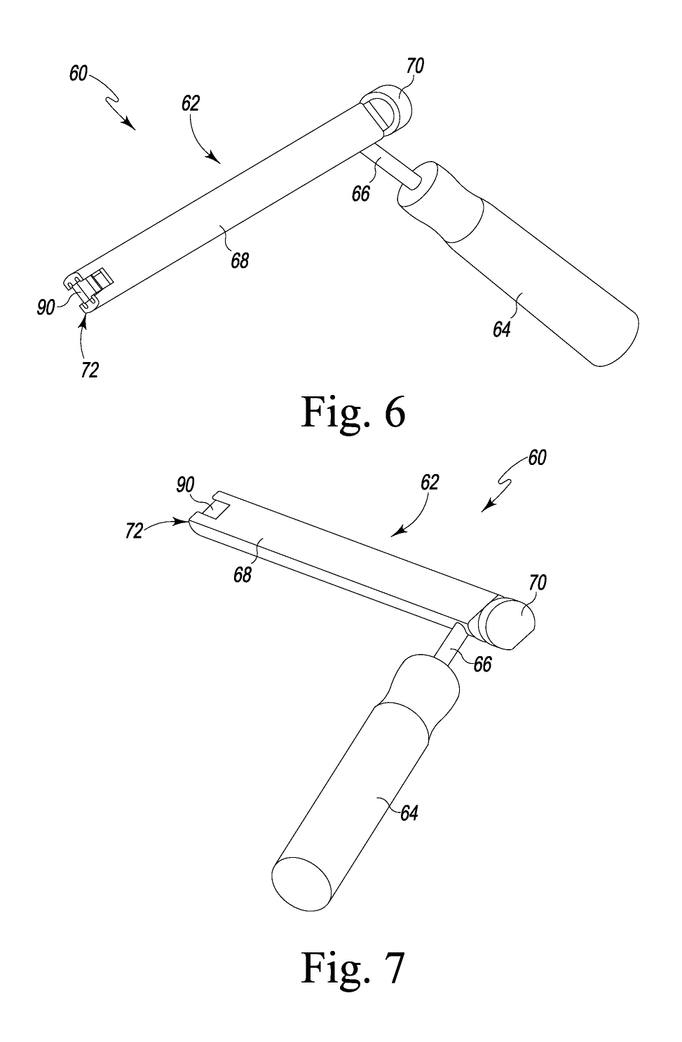

Referring to FIGS. 6-8, there is depicted another exemplary embodiment of an instrument, device or apparatus, generally designated 60, for deploying, placing, installing and/or implanting (collectively, “deployment instrument 60”) the spinal implants of the present invention. The deployment instrument 60 is configured, adapted and/or operable to place, install or implant the staples 14/14a, 16/16a, and 52/54 or similar fasteners of the spinal implants 10/10a/50 or similar spinal implant fashioned in accordance with the present principles, into a vertebrae or an endplate of a vertebrae. The deployment instrument 60 is formed of a suitable material such as a metal and, particularly but not necessarily, a titanium based metal.

The deployment instrument 60 is defined by a handle 64 connected via a neck or stem 66 to a driver 62. The driver 64 carries the staples for implanting while a plunger 70 of the driver 64, aided by an externally applied mallet or the like to the head of the plunger 70, deploys the staples (i.e. implants them into the vertebrae/vertebrae endplate). The driver 64 is defined by a hollow and generally rectangular body or tube 68 that is open at one end to receive the plunger 70, and which has a fastener holding structure 72 on the other end thereof.

The plunger 70 is defined by a generally rectangular rod that is sized to be received in the hollow driver 68. The plunger 70 is axially movable in the driver 68 and relative to the handle 64. The end of the plunger 70 extending from a distal end of the driver 68 (relative to the tip 72) is configured to allow striking thereof for driving the plunger 70 into one or more staples held by the end structure 72. Striking of the exposed end (head) of the plunger 70 while holding the handle 64, axially moves the plunger 70 relative to the handle 64 to impact against a staple for driving the staple into the vertebra/endplate.

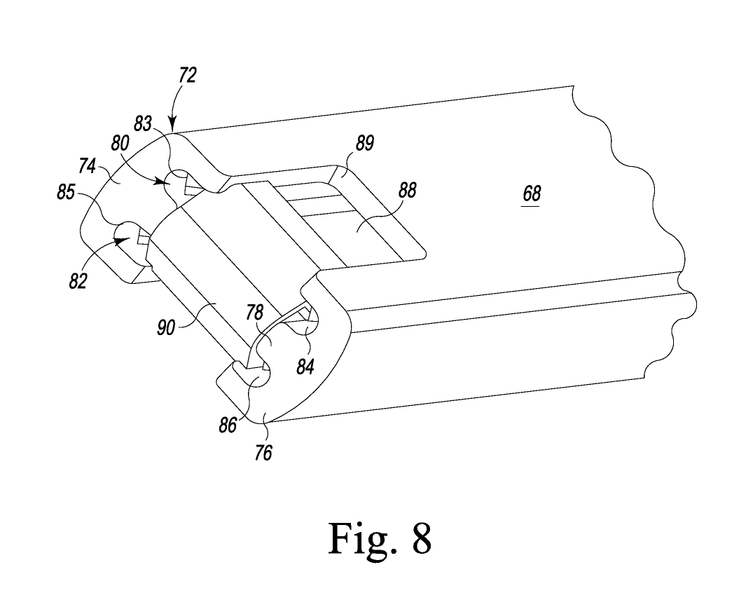

The end structure 72 is configured to install a spinal implant of the present invention (fasteners and covering) onto an intended spinal area. The end structure 72 defines first and second side ends 74, 76 separated by a bar 78. A first elongated opening or slot 80 is situated on one side of the bar 78 while a second elongated opening or slot 84 is situated on another side of the bar 78. For implantation of the spinal implant, a first staple (not shown in FIG. 8) is situated in the opening 80, while a second staple (not shown in FIG. 8) is situated in the opening 82. Since the first and second openings 80, 82 are in communication with the interior of the driver 68, the end 88 of the plunger 70 can impact the staples held therein. The legs of one staple extends from opposite sides 83, 84 of the slot 80 with the legs of the second staple extends from opposite sides 85, 86 of the slot 82 to allow the staple to be driven out of the respective slot 80, 82 by the plunger 70.

As shown in FIG. 8, the end structure 72 also holds a covering 90 of the spinal implant for implanting thereof along with the fasteners. This may be simultaneous. In this manner, the end structure 72 is sized accordingly. The covering 90 extends over the bar 78 with one end thereof situated within a first pocket 89 with the other end thereof situated within a second pocket (not seen in FIG. 8). As can be seen in FIG. 8, the ends of the covering 90 are reduced or configured to allow the legs of the staple in the slot 80, 82 to extend thereabout. During installation, the cross-member of the staple is impacted by the plunger 70 to drive the staple into the vertebra/endplate to thereby hold the end of the covering onto the spinal area.

It should be appreciated that the deployment instrument 50 (preferably, but not necessarily, as well as the deployment instrument 30) is preferably, but not necessarily, configured to fit through a tissue retractor used for microdiscectomy procedures as well as the hemilaminectomy while providing maximum line of sight.

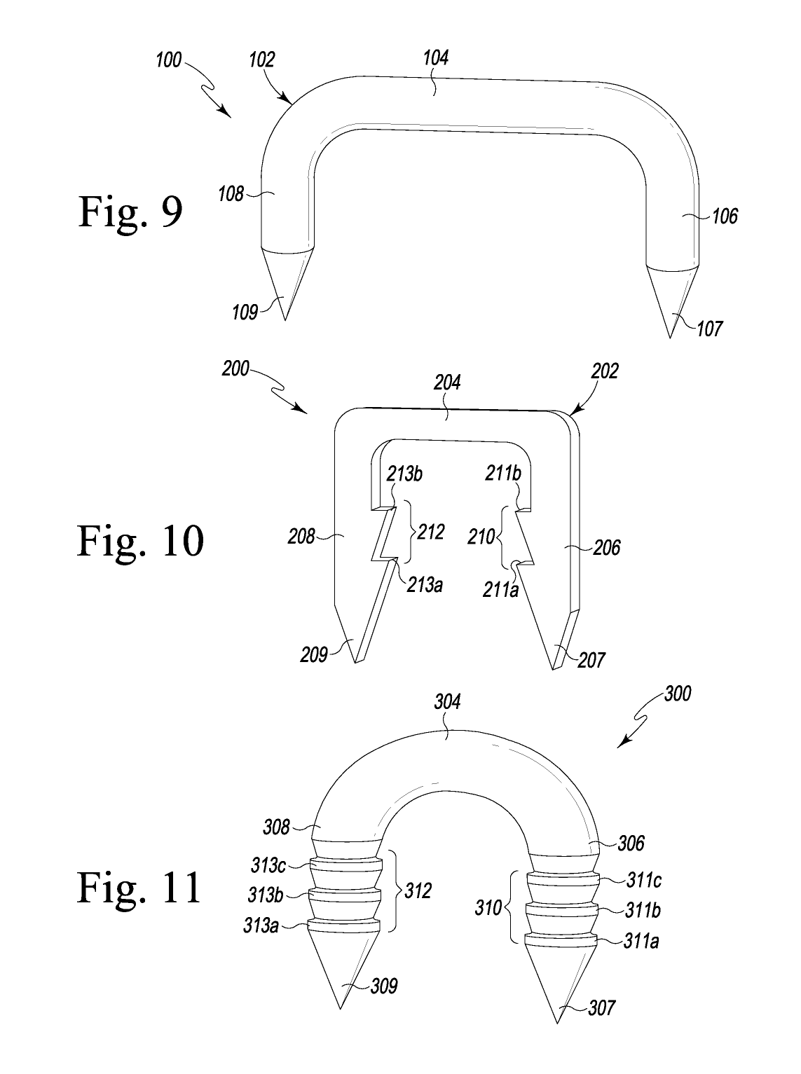

Referring now to FIG. 9, there is depicted another exemplary embodiment of a bone fastener, generally designated 100, for use by the present spinal implants. The bone fastener 100 is fashioned as a bone staple configured for reception and retention in a vertebra. Particularly, the bone staple 100 is fabricated to be attached, anchored, affixed or fastened to one or more vertebral bodies/endplates (vertebrae) as appropriate. The bone staple 100 is made from a biocompatible material such as an implantable grade titanium alloy (e.g. Ti 6Al-4V ELI).

The bone staple 100 is formed as a generally U-shaped member 102 having a generally annular cross section. The bone staple 100 is defined by a crossbar 104 with a first leg 106 and a second leg 108, the first and second legs 106, 108 being generally transverse to the crossbar 104. A first end 107 of the first leg 106 has a conical shaped tip 107 that defines a point. The point of the tip 107 is in the middle of the annulus of the cone so as to be a center point. Likewise, a second end 109 of the second leg 108 has a conical shaped tip that defines a point. The point of the tip 109 is in the middle of the annulus of the cone so as to be a center point. Other configurations are contemplated and capable of use. The first and second legs 106, 108 are spaced a wide width apart and thus the staple 100 may be considered a wide bone staple 100. The outer surface of the legs 106, 108 are smooth, but may include a texture if desired. The staple 100 may also have a coating of a texture, medicament, or mixture thereof. It should be appreciated that the staple 100 is contemplated for use in the present various deployment instruments and spinal implants.

Referring now to FIG. 10, there is depicted another exemplary embodiment of a bone fastener, generally designated 200, for use by the present spinal implants. The bone fastener 200 is fashioned as a bone staple configured for reception and retention in a vertebra. Particularly, the bone staple 200 is fabricated to be attached, anchored, affixed or fastened to one or more vertebral bodies/endplates (vertebrae) as appropriate. The bone staple 200 is made from a biocompatible material such as an implantable grade titanium alloy (e.g. Ti 6Al-4V ELI).

The bone staple 200 is formed as a generally U-shaped member 202 having a generally rectangular cross section. The bone staple 200 is defined by a crossbar 204 with a first leg 206 and a second leg 208, the first and second legs 206, 208 being generally transverse to the crossbar 204. A first end 207 of the first leg 206 has a chisel shaped tip 207 that defines a pointed edge. The pointed edge of the first tip 207 extends the width of the first leg 206 in the direction transverse to the direction of the second leg 208. A second end 209 of the second leg 208 has a chisel shaped tip 209 that defines a pointed edge. The pointed edge of the second tip 209 extends the width of the second leg 208 in the direction transverse to the direction of the first leg 206. Other configurations are contemplated and capable of use. The first and second legs 206, 208 are spaced a narrow width apart and thus the staple 200 may be considered a narrow bone staple 200.

In addition to the features of the bone staple 100, the bone staple 200 further includes leg configurations that aid in insertion and/or anchoring of the bone staple 200 into a vertebra. Particularly, the first leg 206 includes an anchoring element 210, while the second leg 208 includes an anchoring element 212. The anchoring element 210 is formed as one or more steps, ledges, cutouts, notches, protrusions, teeth, serrations, juts or the like, that extend from an inside lateral face or side of the first leg 206 so as to extend toward the second leg 208. By its design, the anchoring element 210 provides easy insertion of the first leg 206 of the bone staple 200 into a vertebral body and resistance to the backing out of the first leg 206 from the vertebral body. The anchoring element 212 is formed as one or more steps, ledges, cutouts, notches, protrusions, teeth, serrations, juts or the like, that extend from an inside lateral face or side of the second leg 208 so as to extend toward the first leg 206. By its design, the anchoring element 212 provides easy insertion of the second leg 208 of the bone staple 200 into a vertebral body and resistance to the backing out of the second leg 208 from the vertebral body.

The outer surface of the legs 206, 208 are smooth, but may include a texture if desired. The staple 200 may also have a coating of a texture, medicament, or mixture thereof. It should be appreciated that the staple 200 is contemplated for use in the present various deployment instruments and spinal implants.

Referring now to FIG. 11, there is depicted another exemplary embodiment of a bone fastener, generally designated 300, for use by the present spinal implants. The bone fastener 300 is fashioned as a bone staple configured for reception and retention in a vertebra. Particularly, the bone staple 300 is fabricated to be attached, anchored, affixed or fastened to one or more vertebral bodies/endplates (vertebrae) as appropriate. The bone staple 300 is made from a biocompatible material such as an implantable grade titanium alloy (e.g. Ti 6Al-4V ELI).

The bone staple 300 is formed as a generally U-shaped member 302 having a generally annular cross section. The bone staple 300 is defined by a crossbar 304 with a first leg 306 and a second leg 308, the first and second legs 306, 308 being generally transverse to the crossbar 304. A first end 307 of the first leg 306 has a conical shaped tip 307 that defines a point. The point of the tip 307 is in the middle of the annulus of the cone so as to be a center point. Likewise, a second end 309 of the second leg 308 has a conical shaped tip that defines a point. The point of the tip 309 is in the middle of the annulus of the cone so as to be a center point. Other configurations are contemplated and capable of use. The first and second legs 306, 308 are spaced a narrow width apart and thus the staple 300 may be considered a narrow bone staple 300.

The bone staple 300, in like manner to the bone staple 200, further includes leg configurations that aid in insertion and/or anchoring of the bone staple 300 into a vertebra. Particularly, the first leg 306 includes an anchoring element 310, while the second leg 308 includes an anchoring element 312. The anchoring element 310 is formed as one or more annular and/or conical or frusto-conical steps, ledges, cutouts, notches, protrusions, teeth, serrations, juts or the like, that extend about the first leg 306. By its design, the anchoring element 310 provides easy insertion of the first leg 306 of the bone staple 300 into a vertebral body and resistance to the backing out of the first leg 306 from the vertebral body. The anchoring element 312 is formed as one or more annular and/or conical or frusto-conical steps, ledges, cutouts, notches, protrusions, teeth, serrations, juts or the like, that extend about the second leg 308. By its design, the anchoring element 312 provides easy insertion of the second leg 308 of the bone staple 300 into a vertebral body and resistance to the backing out of the second leg 308 from the vertebral body.

The outer surface of the legs 306, 308 are smooth, but may include a texture if desired. The staple 300 may also have a coating of a texture, medicament, or mixture thereof. It should be appreciated that the staple 300 is contemplated for use in the present various deployment instruments and spinal implants.

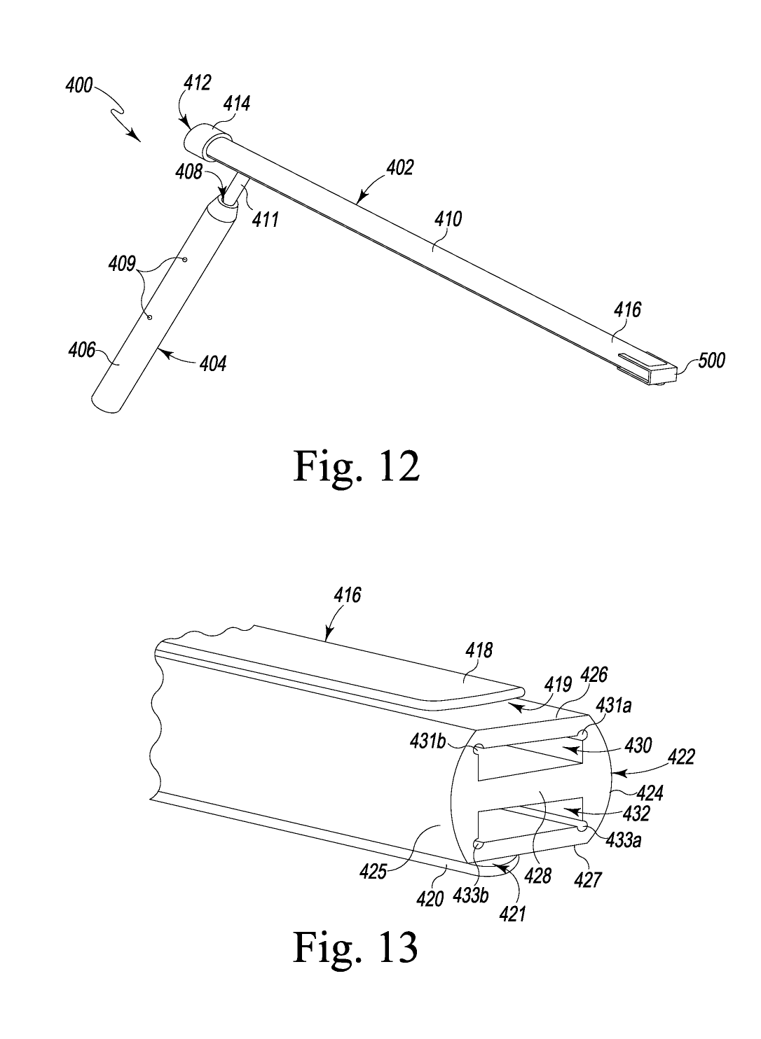

Referring to FIGS. 12-15, there is depicted another exemplary embodiment of an instrument, device or apparatus, generally designated 400, for deploying, placing, installing and/or implanting (collectively, “deployment instrument 400”) the spinal implants of the present invention. The deployment instrument 400 is configured, adapted and/or operable to place, install or implant the bone staples presented herein or similar fasteners of the various spinal implants presented herein or similar spinal implants fashioned in accordance with the present principles, into a vertebral body. The deployment instrument 400 is formed of a suitable material such as a metal and, particularly but not necessarily, a titanium based metal.

The deployment instrument 400 is defined by a driver 402 that is adjustably carried on a handle 404. The driver 402 is defined by a generally tubular body 410 to which is connected a stem or neck 411 formed as a rod, plunger or the like that extends into an elongated bore 408 in the body 406 of the handle 404. Axial movement of the stem 411 and thus the body 410, provide adjustment of the driver 402 relative to the handle 404. Adjustment pegs 409, operatively connected to the stem 411 of the driver 402, extend through the handle body 406 to provide releasable axial adjustment of the driver body 410 relative to the handle 404.

The driver 402 carries bone staples in an axial bore 412 that extends from the tip 424 of the head 416 of the driver body 410 to the distal end 414 of the driver body 410. The bore 412 is configured to accept two bone staples and a staple plunger/driver. The plunger (not seen, but see, e.g. plunger 44 of FIG. 4), aided by an externally applied mallet or the like to the head of the plunger, deploys the staples (i.e. implants them into the vertebral body). The driver 402 is thus defined by a hollow and generally oblate, ovoid, annular, rectangular or otherwise shaped body 410 that is open at one end (414) to receive the plunger, and which has a fastener holding structure/tip 422 on the other end (416) thereof. The plunger is configured and operates in the same manner as plunger 70. Other configurations and operation may be used.

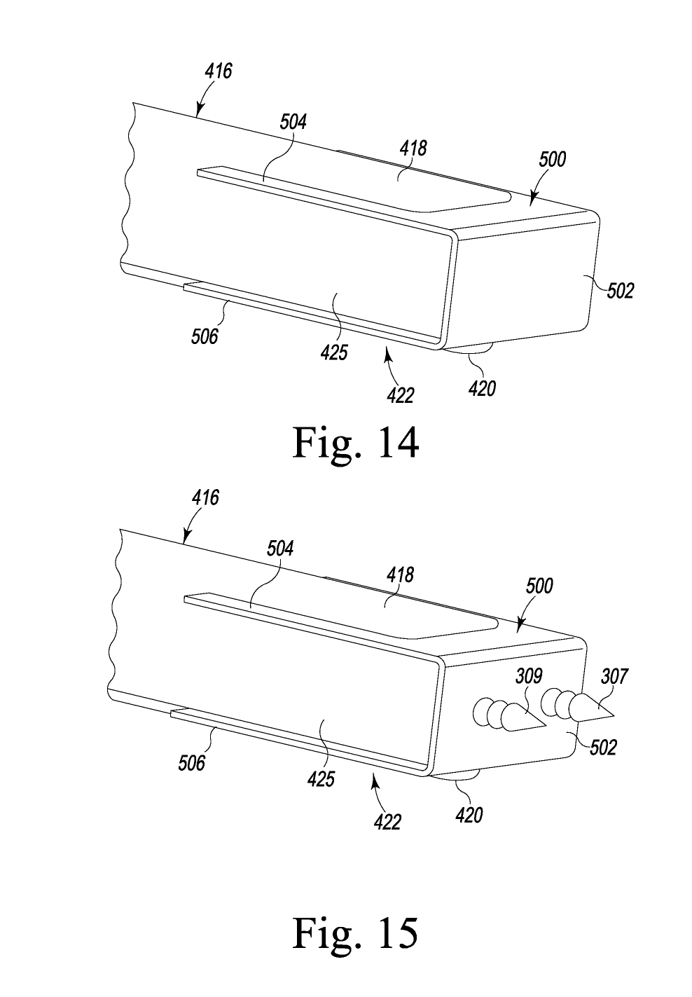

The tip 422 of the end 416 is configured to install a spinal implant of the present invention (fasteners and covering) onto and/or over an intended spinal implant site or area. The end 416 is thus configured to hold and temporarily retain a covering and covering fasteners (the present spinal implant) while the spinal implant is being installed on the spine. As such, the end 416 has a first tang 418 that extends axially along the outer surface of a side of the end 416 of the body 410, and a second tang 420 that extends axially along the outer surface of another side of the end 416 of the body 410. Preferably, and as shown, but not necessarily, the tangs 418, 420 are disposed on opposite sides of the end 416. The first tang 418 defines an open pocket or area 419 between the end 416 and the first tang 418, while the second tang 420 defines an open pocket or area 421 between the end 416 and the second tang 420. The pockets 419, 421 provide covering retention areas for retaining ends of a covering while the covering is being installed such as shown in FIG. 12 (see also FIG. 14 where the ends 504 and 506 of the covering 500 are held by the first and second tangs 418, 420). The covering 500 is thus held onto the tip 422 of the instrument 400 while the spinal implant is being installed.

The end 416 holds one or two bone staples for fastening the covering 500 onto a vertebra. As such, the tip 422 has a first elongated opening, bore or slot 430 and a second elongated opening, bore or slot 432 each of which is in communication with the bore 412 of the driver 402 and spaced from each other so as to define a middle portion or face 428. The slots 430, 432 allow the reception and retention of a bone staple such as one of those presented herein. The tip 422 is generally rectangular/ovoid in shape to define a first rounded side 424, a second rounded side 425 opposite to the first rounded side 424, a first flat side 426, and a second flat side 427 opposite to the first flat side 426. For implantation of the spinal implant, a first staple (not shown in FIG. 13) is situated in the opening 430, while a second staple (not shown in FIG. 13) is situated in the opening 432. Since the first and second openings 430, 432 are in communication with the interior of the driver 402, the end of the plunger can impact the staples held therein.

The legs of one staple extends from opposite sides of the slot 430, with the legs of the second staple extends from opposite sides of the slot 432 to allow the staple to be driven out of the respective slot. The slot 430 includes a rounded groove or channel 431a on a first outer edge thereof, and a rounded groove or channel 431b on a second outer edge thereof. Likewise, the slot 432 includes a rounded groove or channel 433a on a first outer edge thereof, and a rounded groove or channel 433b on a second outer edge thereof. During installation, and as shown in FIG. 14, a covering 500 is situated on the tip 422 of the instrument 400 ready for installation. As shown in FIG. 15, ends 307, 309 of the staple 300 are protruding from a face, side or area 502 of the covering 500 waiting to be driven into the vertebral body for anchoring the covering 500 to the spinal implant site or area.

It should be appreciated that the deployment instrument 400 (preferably, but not necessarily, as well as the other deployment instruments herein) is preferably, but not necessarily, configured to fit through a tissue retractor used for microdiscectomy procedures as well as the hemilaminectomy while providing maximum line of sight.

It should be appreciated that the above figures and descriptions are only exemplary of the many spinal implant (fastener and covering) configurations and dimensions possible in accordance with the present principles.

While this invention has been described as having preferred designs, the present invention can be further modified within the spirit and scope of this disclosure. This application is therefore intended to cover any variations, uses, of adaptations of the invention using its general principles. Further, this application is intended to cover such departures from the present disclosure as come within known or customary practice in the art to which this invention pertains.

What is claimed is:

1. A spinal implant for application to a spinal disc area via a spinal implant application instrument, the spinal implant comprising:
a covering formed of a resilient material and configured to be retained by a spinal implant application instrument for installation thereof;
a first fastener associated with the resilient material and configured to penetrate and anchor itself to a vertebral body and retain a portion of the covering to the spinal disc area, the first fastener configured to be provided via the spinal implant application instrument; and
a second fastener associated with the resilient material and configured to penetrate and anchor itself to a vertebral body and retain a portion of the covering to the spinal disc area, the second fastener configured to be provided via the spinal implant application instrument.

2. The spinal implant of claim 1, wherein the resilient material comprises an elastic material.

3. The spinal implant of claim 2, wherein the first and second fasteners are bone staples.

4. The spinal implant of claim 3, wherein the bone staples are defined by first and second legs connected via a crossbar, the first and second legs each having a tip configured to penetrate into the vertebral body.

5. The spinal implant of claim 4, wherein the first leg includes a first leg structure configured to anchor itself to the vertebral body, and the second leg includes a second leg structure configured to anchor itself to the vertebral body.

6. The spinal implant of claim 5, wherein the first and second leg structures each include a plurality of teeth.

7. The spinal implant of claim 5, wherein the first and second leg structures each include a plurality of annular ledges.

8. The spinal implant of claim 7, wherein each plurality of annular ledges define a plurality of frusto-conical portions.

9. A spinal implant for application to a portion of a spinal disc via a spinal implant application instrument, the spinal implant comprising:
a mesh formed of an elastic material and configured to be retained on an end of a spinal implant application instrument for installation thereof;
a first bone staple configured to penetrate and anchor itself to a vertebral body and retain a portion of the mesh to the vertebral body, the first bone staple provided via the spinal implant application instrument; and
a second bone staple configured to penetrate and anchor itself to a vertebral body and retain a portion of the mesh to the vertebral body, the second bone staple provided via the spinal implant application instrument.

10. The spinal implant of claim 9, wherein the bone staples are defined by first and second legs connected via a crossbar, the first and second legs each having a tip configured to penetrate into the vertebral body.

11. The spinal implant of claim 10, wherein the first leg includes a first leg structure configured to anchor itself to the vertebral body, and the second leg includes a second leg structure configured to anchor itself to the vertebral body.

12. The spinal implant of claim 11, wherein the first and second leg structures each include a plurality of teeth.

13. The spinal implant of claim 11, wherein the first and second leg structures each include a plurality of annular ledges.

14. The spinal implant of claim 13, wherein each plurality of annular ledges define a plurality of frusto-conical portions.

15. The spinal implant of claim 9, wherein the spinal implant is part of a kit with the spinal implant application instrument.

16. A spinal implant comprising:
an elastic mesh;
a first bone staple having first and second legs extending from a cross member, the first and second legs each having a pointed end configured to penetrate into a vertebral body, the cross member configured to retain a portion of the elastic mesh to the vertebral body; and
a second bone staple having first and second legs extending from a cross member, the first and second legs each having a pointed end configured to penetrate into a vertebral body, the cross member configured to retain a portion of the elastic mesh to the vertebral body.

17. The spinal implant of claim 16, wherein each first leg includes a first leg structure configured to anchor itself to the vertebral body, and each second leg includes a second leg structure configured to anchor itself to the vertebral body.

18. The spinal implant of claim 17, wherein each first and second leg structure includes a plurality of teeth.

19. The spinal implant of claim 17, wherein each first and second leg structure each includes a plurality of annular ledges.

20. The spinal implant of claim 19, wherein each plurality of annular ledges define a plurality of frusto-conical portions.
